(19) United States
(12) Patent Application Publication
(10) Pub. No.: US 2009/0023754 A1
(43) Pub. Date: Jan. 22, 2009

(54) Modified release famciclovir pharmaceutical compositions

(76) Inventors: Wai Yip Lee, Bedminster, NJ (US); Shoufeng Li, Basking Ridge, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/920,099

(22) PCT Filed: May 8, 2006
(86) PCT No.: PCT/US2006/017709

§ 371 Date: Nov. 8, 2007

Related U.S. Application Data
(60) Provisional application No. 60/679,606, filed on May 10, 2005.

(51) Int. Cl. A61K 31/52

(52) U.S. Cl. 514/263.4

(57) ABSTRACT

A modified release pharmaceutical composition of famciclovir contains at least 60% by weight famciclovir with at least 5% by weight of a release retardant. Particularly useful as a release retardant include polymers, especially a mixture of polyvinyl acetate and polyvinylpyrrolidone. A method of making such pharmaceutical compositions using a extruder and a granulation method is particularly useful.

### FIELD OF THE INVENTION

The present invention relates to a novel pharmaceutical compositions of famciclovir, especially modified release compositions.

### BACKGROUND OF THE INVENTION

Famciclovir, or the compound 2-[2-(2-amino-9H-purin-9-yl)ethyl]-1,3-propanediol acetate, is an orally administered prodrug of the antiviral agent penciclovir. Penciclovir has inhibitory activity against herpes simple virus type1 (HSV-1), type 2 (HSV-2) and varicella zoster virus (VZV).

As currently marketed, famciclovir is administered in the form of immediate release tablets containing 125, 250, 500 and 750 mg of the therapeutic compound. The tablets contain conventional excipients used in solid oral dosage forms, e.g., lactose, sodium starch glycolate and magnesium stearate. The tablets are administered up to three times a day.

Famciclovir is rapidly absorbed after oral administration of an immediate release formulation. This may be due to its high solubility which in turns gives rise to a rapid absorption. At the same time, its relatively short half-life contributes to its rapid elimination from the plasma. As a result, in order to maintain a therapeutic level of famciclovir for an extended period of time, patients may need to take up to three times a day. In such a case, patient compliance may be a problem. In addition, some adverse reactions such as nausea may be related to the high Cₘₐₓ of the immediate release dosage form.

Modified release dosage forms, can deliver the therapeutic compound in a safe and effective range over a longer period of time than a regular immediate release dosage form by lowering peak to trough ratio, thereby allowing less frequent dosing. Thus, a patient is more likely to comply with the regimen prescribed by physician.

Despite the merits described above, it is difficult to develop a modified release formulation for famciclovir because of high therapeutic compound loading required in such a formulation.

U.S. Pat. No. 6,765,007 ('007 patent), which is hereby incorporated by reference in its entirety, discloses immediate release tablets containing famciclovir wherein the percentage of famciclovir by weight in the tablet is greater than or equal to 85%. For example, tablets include famciclovir along with hydroxypropyl cellulose, sodium starch glycolate, magnesium stearate and anhydrous lactose. However, modified release formulations of famciclovir are not disclosed in the '007 patent. Therefore, there is a need for a method of administering famciclovir in an oral dosage form once per day that provides therapeutically effective plasma concentrations of famciclovir. The present invention addresses this need.

### SUMMARY OF THE INVENTION

In a first aspect, the present invention features a modified release pharmaceutical composition that includes a therapeutic compound, e.g., famciclovir, and a release retardant.

In a particular aspect of the present invention, the pharmaceutical composition includes at least 60% by weight famciclovir and at least 5% by weight release retardant. The release retardant, e.g., can be a water soluble, water-swellable or water insoluble polymer and mixtures thereof. In another aspect of the present invention, the polymer has a glass transition temperature less than the melting range of famciclovir. Particularly useful as the release retardant in the present invention is a mixture of polyvinylacetate and polyvinylpyrrolidone.

In another aspect of the present invention, the release retardant is a non-polymeric hydrophobic release retardant. The non-polymeric hydrophobic release retardant, for example, has a melting point less than the melting range of famciclovir.

The present inventions also includes methods of manufacturing the modified release pharmaceutical compositions. For example, granulating of the therapeutic compound and the release retardant can be accomplished by the use of an extruder. The resulting granules can constitute an internal phase for subsequent processing, for example direct compression into a tablet or encapsulation by a capsule.

### BRIEF DESCRIPTION OF THE DRAWINGS

The accompanying drawing, which is incorporated in and constitutes a part of the specification, illustrates exemplary embodiments of the present invention.

FIG. 1 shows a chart depicting the dissolution profiles for exemplary embodiments in accordance with the present invention as disclosed in Examples 1, 2, 3, 4 and 5.

### DETAILED DESCRIPTION OF THE INVENTION

As used herein, the term “pharmaceutical composition” means a mixture or solution containing a therapeutic compound to be administered to a mammal, e.g., a human in order to prevent, treat or control a particular disease or condition affecting the mammal.

As used herein, the term “therapeutic compound” means any compound, substance, drug, medicament, or active ingredient having a therapeutic or pharmacological effect, and which is suitable for administration to a mammal, e.g., a human, in a composition that is particularly suitable for oral administration.

Examples of therapeutic classes of therapeutic compounds include, but are not limited to, antihypertensives, antianxiety agents, anticlotting agents, anticonvulsants, blood glucose-lowering agents, decongestants, antihistamines, antitussives, antineoplastics, beta blockers, anti-inflammatories, antipsychotic agents, cognitive enhancers, anti-atherosclerotic agents, cholesterol reducing agents, antiobesity agents, autoimmune disorder agents, anti-impotence agents, antibacterial and antifungal agents, hypnotic agents, antibiotics, anti-depressants, antiviral agents and combinations of the foregoing.

The therapeutic compound(s) is present in the pharmaceutical compositions of the present invention in a therapeutically effective amount or concentration. Such a therapeutically effective amount or concentration is known to one of ordinary skill in the art as the amount or concentration varies with the therapeutic compound being used and the indication which is being addressed. For example, in accordance with the present invention, the therapeutic compound may be present in an amount by weight from about 60% to about 95% by weight of the pharmaceutical composition; e.g., from about 90% to about 95% by weight of the pharmaceutical composition.

A therapeutic compound of particular interest for use in the present invention is famciclovir which is 2-[2-(2-amino-9H-purin-9-yl)ethyl]-1,3-propanediol diacetate. Famciclovir is disclosed as Example 2 in U.S. Pat. No. 5,250,688, which is hereby incorporated by reference. Famciclovir has the following structure:

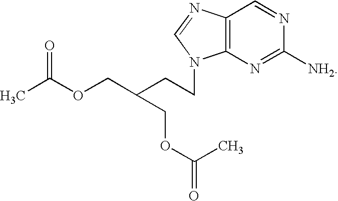

Famciclovir, a crystalline compound, has a relatively low melting range of about 102° C. to about 104° C. (i.e., approximately 104° C.), and is highly water soluble with a solubility about 300 mg/mL in an acidic medium and about 22 mg/mL in a basic medium. As used herein, the term “melting range” refers to the range of temperatures from the lower temperature at which the first drop of liquid begins to form from the solid phase to the higher temperature at which the entire mass of solid material becomes a liquid material.

As used herein, the term “immediate release” refers to formulations or dosage units that rapidly dissolve in vitro and are intended to be completely dissolved and absorbed in the stomach or uppers gastrointestinal tract. For example, immediate release formulations release at least 90% of the therapeutic compound within 30 minutes of administration.

In contrast, as used herein, the term “modified release” refers to formulations or dosage units of the present invention that are slowly and continuously dissolved and absorbed in the stomach and gastrointestinal tract over a period of time about two hours or more. Controlled-release may also refer to delayed-release in which release of the therapeutic compound does not start immediately when the pharmaceutical composition reaches the stomach but is delayed for a period of time, for instance, until when the pharmaceutical composition reaches the intestine when the increasing pH is used to trigger release of the therapeutic compound from the pharmaceutical composition. A modified release profile for the present invention can be a zero order release profile.

As used herein, the term “release retardant” refers to any material or substance that slows the release of a therapeutic compound from a pharmaceutical composition when orally ingested. Various sustained release systems, as known in the art, can be accomplished by the use of a release retarding component, e.g., a diffusion system, a dissolution system and/or an osmotic system. A release retardant can be polymeric or non-polymeric in nature. The pharmaceutical compositions of the present invention include at least 5% of a release retardant by weight of the composition.

As used herein, the term “polymer” or “polymeric” refers to a polymer or mixture of polymers that have a glass transition temperature (T_{g}) or softening temperature less than or approximately equal to the melting point (or melting range) of famciclovir. The glass transition temperature is the temperature at which the polymer's characteristics change from that of highly viscous to that of relatively less viscous mass. Types of polymers include, but are not limited to, water-soluble, water-swellable, water insoluble polymers and combinations of the foregoing. A plasticizer, can optionally be used with the polymer in order to lower that polymer's glass transition temperature in the event that the polymer's T_{g} exceeds that of famciclovir's melting range and to render that polymer suitable for use in the present invention.

As used herein, the term “plasticizer” refers to a material that may be incorporated into the pharmaceutical composition in order to decrease the T_{g} and the melt viscosity of a polymer by increasing the free volume between polymer chains. Plasticizers, e.g., include, but are not limited to, water; citrate esters, e.g., triethylcitrate, triacetin; low molecular weight poly(alkylene oxides), e.g., poly(ethylene glycols), poly(propylene glycols), poly(ethylene/propylene glycols), glycerol, pentaerythritol, glycerol monoacetate, diacetate or triacetate; propylene glycol; sodium diethyl sulfosuccinate; and the therapeutic compound itself. The plasticizer can be present in concentration from about 0-15%, e.g., 0.5-5% by weight of the pharmaceutical composition. Examples of plasticizers can also be found in The Handbook of Pharmaceutical Additives, Ash et al., Gower Publishing (2000).

Examples of polymers include, but are not limited to,
homopolymers and copolymers of N-vinyl lactams, e.g., homopolymers and copolymers of N-vinyl pyrrolidone (e.g., polyvinylpyrrolidone), copolymers of N-vinyl pyrrolidone and vinyl acetate or vinyl propionate;
cellulose esters and cellulose ethers (e.g., methylcellulose and ethylcellulose) hydroxyalkylcelluloses (e.g., hydroxypropylcellulose), hydroxyalkylalkylcelluloses (e.g., hydroxypropylmethylcellulose), cellulose phthalates (e.g., cellulose acetate phthalate and hydroxylpropylmethylcellulose phthalate) and cellulose succinates (e.g., hydroxypropylmethylcellulose succinate or hydroxypropylmethylcellulose acetate succinate);
high molecular polyalkylene oxides, such as polyethylene oxide and polypropylene oxide and copolymers of ethylene oxide and propylene oxide;
polyacrylates and polymethacrylates (e.g., methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates));
polyacrylamides;
vinyl acetate polymers, such as copolymers of vinyl acetate and crotonic acid, partially hydrolyzed polyvinyl acetate;
polyvinyl alcohol; and
oligo- and polysaccharides, such as carrageenans, galactomannans and xanthan gum, or mixtures of one or more thereof.

Of the aforementioned polymeric materials, particularly useful is a mixture of polyvinyl acetate and polyvinylpyrrolidone, e.g., in a ratio of about 4:1. Such a polymeric material is commercially available as KOLLIDON SR from BASF AG (Ludwigshafen, Germany).

As used herein, the term “non-polymeric release retardant” refers to substances or a mixtures of substances, non-polymeric in nature, that are solid or semi-solid at room temperature (about 25° C.) and with melting points (or melting ranges) less than or approximately equal to the melting range of famciclovir.

Particularly useful as non-polymeric release retardants are hydrophobic non-polymeric release retardants. As used herein, the term “hydrophobic”, with respect to the release retardant, refers to being more compatible with oil than with water. A substance with hydrophobic properties is insoluble or almost insoluble in water but is easily soluble in oil or other non-polar solvents.

Examples of hydrophobic non-polymeric release retardants include, but are not limited to, esters, hydrogenated oils, natural waxes, synthetic waxes, hydrocarbons, fatty alcohols, fatty acids, monoglycerides, diglycerides, triglycerides and mixtures thereof.

Examples of esters, such as glyceryl esters include, but are not limited to, glyceryl monostearate, e.g., CAPMUL GMS from Abitec Corp. (Columbus, Ohio); glyceryl palmitostearate, e.g., PRECIROL ATO 5 (melting range from 53-57° C.) from Gattefossé, S. A. (St. Priest, France); glyceryl behenate, e.g., COMPRITOL ATO 888 (melting range from 69-74° C.) from Gattefossé, S. A.; lauroyl macrogol glycerides, e.g., GELUCIRE 44/14 (melting range from 43-48° C.) from Gattefossé, S. A.; stearoyl macrogol glycerides, e.g., GELUCIRE 50/13 (melting range from 46-51° C.) from Gattefossé, S. A.; and acetylated glycerol monostearate; sorbitan monostearate, e.g., ARLACEL 60 from Uniqema (New Castle, Del.); and cetyl palmitate, e.g., CUTINA CP from Cognis Corp. (Dusseldorf, Germany).

Examples of hydrogenated oils include, but are not limited to, hydrogenated castor oil, e.g., CUTINA HR from Cognis Corp; hydrogenated cottonseed oil; hydrogenated soybean oil; and hydrogenated palm oil.

Examples of waxes include, but are not limited to, carnauba wax, beeswax and spermaceti wax.

Examples of hydrocarbons include, but are not limited to, microcrystalline wax and paraffin.

Examples of fatty alcohols, i.e., higher molecular weight nonvolatile alcohols that have from about 14 to about 31 carbon atoms include, but are not limited to, cetyl alcohol, e.g., CRODACOL C-70 from Croda Corp. (Edison, N.J.); stearyl alcohol, e.g., CRODACOL S-95 from Croda Corp; lauryl alcohol; and myristyl alcohol. Examples of fatty acids which may have from about 10 to about 22 carbon atoms include, but are not limited to, stearic acid, e.g., HYSTRENE 5016 from Crompton Corp. (Middlebury, Conn.); decanoic acid; palmitic acid; lauric acid; and myristic acid.

A particularly useful hydrophobic non-polymeric release retardant in the present invention is glyceryl behenate, or COMPRITOL ATO 888.

As used herein, the term “melt granulation” refers to an exemplary process of manufacturing the modified release pharmaceutical compositions of the present invention whereby the processing is accomplished by the use of an extruder. The processing temperatures as used in the extruder do not exceed the melting point or melting range of famciclovir.

In general, an extruder includes a rotating screw(s) within a stationary barrel with an optional die located at one end of the barrel. Along the entire length of the screw, distributive mixing of the materials (e.g., the therapeutic compound, release retardant, and any other needed excipients) is provided by the rotation of the screw(s) within the barrel. Conceptually, the extruder can be divided into three sections: a feeding section; a heating section and a metering section. In the feeding section, the raw materials are fed into the extruder, e.g., from a hopper. The raw materials can be directly added to the hopper without the need of a solvent. In the heating section, the raw materials are heated to a temperature less than the melting range of famciclovir but greater than the T_{g} of the retardant and/or melting temperature of the non-polymeric release retardant. After the heating section is a metering section in which the mixed materials are optionally extruded through a die into a particular shape, e.g., granules or noodles. Types of extruders particularly useful in the present invention are single- and twin-screw extruders. Such equipment and techniques used to make pharmaceutical composition by extrusion have been established and are well-known in the prior art. See, e.g., Breitenbach, Eur J Pharma Biopharma, Vol. 54, pp. 107-17 (2002), which is hereby incorporated by reference in its entirety. See also, e.g., U.S. Pat. Nos. 4,801,460; 5,456,923; 5,700,410; and 5,945,127.

The manufacturing of the pharmaceutical compositions of the present invention begins with the compounding of the therapeutic compound along with the release retardant using melt granulation to form an extrudate. The release retardant, e.g., may be present in an amount from about 5% to about 40% by weight of the composition of the extrudate, e.g., from about 10% to about 35%, e.g., from about 25% to about 30%. Similarly, the therapeutic compound, may be present in an amount from about 60% to about 99% by weight of the composition of the extrudate, e.g., from about 70% to about 90%, e.g., from about 80% to about 85%.

The extrudate is, e.g., subsequently milled into granules which form the internal phase of the pharmaceutical composition. One of ordinary skill in the art will appreciate the necessary particle size of the granule that is necessary for the particular pharmaceutical composition being formulated. For example, suitable particle sizes, include those of less than equal to 1,000 μm, 750 μm, 500 μm or 250 μm. Alternatively, the extrudate can be directly molded into tablets, cut into multiparticles or processed into any other forms as known to one of ordinary skill in the art.

The resulting granules are, e.g., particles of the therapeutic compound embedded, substantially embedded in, coated, continuously or discontinuously, by the release retardant.

The granules may be formulated into oral forms, e.g., solid oral dosage forms, such as tablets, pills, lozenges, caplets, capsules or sachets. Such oral dosage forms may comprise conventional pharmaceutical excipients. Examples of such excipients include, but are not limited to, disintegrants, plasticizers, binders, lubricants, glidants, stabilizers, and diluents. Any release retardants known to one of ordinary skill in the art, including the release retards aforementioned, can also be added. One of ordinary skill in the art may select one or more of the aforementioned excipients with respect to the particular desired properties of the solid oral dosage form by routine experimentation and without any undue burden. The amount of each excipient used may vary within ranges conventional in the art. The following references which are all hereby incorporated by reference discloses techniques and excipients used to formulate oral dosage forms. See The Handbook of Pharmaceutical Excipients, 4^{th} edition, Rowe et al., eds., American Pharmaceuticals Association (2003); and Remington: the Science and Practice of Pharmacy, 20^{th} edition, Gennaro, ed., Lippincott Williams & Wilkins (2003). The granules can be combined with the excipients by using, e.g., a V-blender. Subsequent processing can include compression or molding into a tablet or encapsulating into a capsule.

Examples of pharmaceutically acceptable disintegrants include, but are not limited to, starches; clays; celluloses; alginates; gums; cross-linked polymers, e.g., cross-linked polyvinyl pyrrolidone or crospovidone, e.g., POLYPLASDONE XL from International Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. The disintegrant, e.g., may be present in an amount from about 0% to about 45% by weight of the composition; e.g., from 0% to about 10%. In an exemplary embodiment of the present invention, no disintegrant is used in the formulation resulting in a monolithic solid dosage form.

Examples of pharmaceutically acceptable binders include, but are not limited to, starches; celluloses and derivatives thereof, for example, microcrystalline cellulose, e.g., AVICEL PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatin. The binder, e.g., may be present in an amount from about 0% to about 45% by weight of the composition; e.g., from 0% to about 10%.

Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant and/or glidant, e.g., may be present in an amount from about 0% to about 45% by weight of the composition; e.g., from 0% to about 10%.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 0% to about 45% by weight of the composition; e.g., from 0% to about 10%.

The therapeutic compound and the release retardant are mixed in a ratio of release retardant to therapeutic compound in a range of 1:1.5 to 1:1 to 1:19 (on a dry weight basis), or more particularly in a range of 1:1 to 1:8, e.g., 1:1 to 1:4 (on a dry weight basis) in a extruder, e.g., a twin screw extruder to form an extrudate. While in the extruder, the materials are heated to a temperature below the melting range of the therapeutic compound but greater than melting point of the non-polymeric release retardant (if present) and/or glass transition temperature of the polymeric release retardant (if present). The mixture is optionally extruded through a die to form an extrudate. After cooling, the extrudate can be milled into granules and subsequently screened through a sieve.

Once the tablets are obtained, they can be optionally coated with a functional or non-functional coating as known in the art. Examples of coating techniques include, but are not limited to, sugar coating, film coating, microencapsulation and compression coating. Types of coatings include, but are not limited to, enteric coatings, sustained release coatings, controlled-release coatings.

The utility of all the pharmaceutical compositions of the present invention may be observed in standard clinical tests in, e.g., known indications of drug dosages giving therapeutically effective blood levels of the therapeutic compound, e.g., using dosages in the range of 2.5-1000 mg of therapeutic compound per day for a 75 kg mammal, e.g., adult and in standard animal models.

The pharmaceutical composition, e.g., in form of a tablet or a powder suitable for tablet formulation will suitably contain between 250 mg and 1,500 mg of the therapeutic compound, e.g., 500, 750 or 1000 mg. Such unit dosage forms are suitable for administration one to two times daily depending upon the particular purpose of therapy, the phase of therapy and the like.

The present invention provides a method of treatment of a subject suffering from a disease, condition or disorder treatable with a therapeutic compound comprising administering a therapeutically effective amount of a pharmaceutical composition of the present invention to a subject in need of such treatment. Additionally, the present invention provides the use of a composition according to the present invention comprising famciclovir in the manufacture of a medicament for the treatment and/or prevention of conditions, such as HSV-1, HSV-2 and VZV.

Once formulated into a final oral dosage form, the modified release pharmaceutical compositions can have the following exemplary dissolution profiles. For example, less than 50% of the therapeutic compound release in a half hour with the balance released over a time period from 4-24 hours. Alternatively, less than 50% release in a half hour and equal to or less than a 100% released in four hours.

The following examples are illustrative, but do not serve to limit the scope of the invention described herein. The examples are meant only to suggest a method of practicing the present invention.

Quantities of ingredients, represented by percentage by weight of the pharmaceutical composition, used in each example are set forth in the respective tables located after the respective descriptions.

### EXAMPLE 1

| | | |
|---|---|---|
| Ingredient | Percentage (w/w) | Amount per tablet (mg) |

Internal phase

| | | |
|---|---|---|
| Famciclovir | 68.9% | 750 |
| PVA/PVP blend | 29.6% | 322 |
| Silicon dioxide | 0.5% | 5.3 |

External phase

| | | |
|---|---|---|
| Magnesium stearate | 1% | 10.9 |
| Total | | 1088.2 |

The internal phase ingredients: famciclovir, PVA/PVP blend commercially-available as KOLLIDON SR from BASF AG (Ludwigshafen, Germany), and silicon dioxide are screened using an #18 mesh screen (i.e., a one mm screen), and a pre-blend is prepared. The internal phase is then introduced into the feed section, or hopper, of a twin screw extruder. A suitable twin screw extruder is the PRISM 16 mm pharmaceutical twin screw extruder available from Thermo Electron Corp. (Waltham, Mass.).

The twin screw extruder is configured with four individual barrel zones, or sections without the fifth zone (i.e., the die). Starting from the hopper, the zones are respectively heated to the following temperatures: 90° C., 90° C., 60° C. and 40° C. As the material progresses through the extruder, the speed of the screws is gradually increased to 150 rpm.

The extrudate, or granules, from the extruder are then cooled to room temperature. Subsequent to cooling, the extrudate is milled to a size less than 300 hundred microns.

For the external phase, the magnesium stearate is first passed through an 18 mesh screen. The magnesium stearate is then blended with the obtained granules from the internal blender in a bin blender for approximately 60 rotations. The resulting final blend is compressed into tablets using a conventional rotary tablet press (e.g., Manesty Beta Press). The resulting tablets are monolithic.

### EXAMPLE 2

| | | |
|---|---|---|
| Ingredient | Percentage (w/w) | Amount per tablet (mg) |

Internal phase

| | | |
|---|---|---|
| Famciclovir | 78.8% | 750 |
| PVA/PVP blend | 19.7% | 187.5 |
| Silicon dioxide | 0.5% | 4.8 |

External phase

| | | |
|---|---|---|
| Magnesium stearate | 1% | 9.5 |
| Total | | 951.8 |

Example 2 is made using the same process as disclosed in Example 1, however, with different concentrations of ingredients.

### EXAMPLE 3

| | | |
|---|---|---|
| Ingredient | Percentage (w/w) | Amount per tablet (mg) |

Internal phase

| | | |
|---|---|---|
| Famciclovir | 88.7% | 750 |
| PVA/PVP blend | 9.8% | 83.7 |
| Silicon dioxide | 0.5% | 4.2 |

External phase

| | | |
|---|---|---|
| Magnesium stearate | 1% | 8.5 |
| Total | | 846.4 |

Example 3 is made using the same process as disclosed in Example 1, however, with different concentrations of ingredients.

### EXAMPLE 4

| | | |
|---|---|---|
| Ingredient | Percentage (w/w) | Amount per tablet (mg) |

Internal phase

| | | |
|---|---|---|
| Famciclovir | 78.8 % | 750 |
| PVA/PVP blend | 9.8% | 93.8 |
| Silicon dioxide | 0.5% | 4.7 |
| Glyceryl behenate | 9.9% | 93.7 |

External phase

| | | |
|---|---|---|
| Magnesium stearate | 1% | 9.5 |
| Total | | 951.7 |

Example 4 is made using the same process as disclosed in Example 1; however, glyceryl behenate is added to the internal phase.

### EXAMPLE 5

| | | |
|---|---|---|
| Ingredient | Percentage (w/w) | Amount per tablet (mg) |

Internal phase

| | | |
|---|---|---|
| Famciclovir | 78.8% | 750 |
| Ethyl cellulose | 9.9% | 93.8 |
| Glyceryl behenate | 9.9% | 93.7 |
| Silicon dioxide | 0.4% | 4.7 |

External phase

| | | |
|---|---|---|
| Magnesium stearate | 1% | 9.5 |
| Total | | 951.7 |

Example 5 is made using the same process as disclosed in Example 1; however, glyceryl behenate and ethylcellulose are substituted for the PVA/PVP blend.

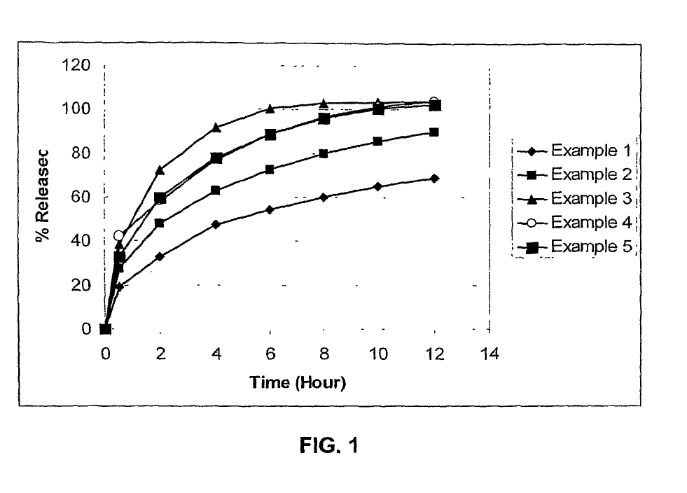

FIG. 1 is a chart showing the dissolution profiles for tablets for each of the five examples. The tablets are placed in 0.1 N HCl using USP Apparatus II rotating at 100 rpm and at 37° C. The chart shows that the examples of the present invention indeed have a sustained release profile. The Y-axis of FIG. 1 represents the percentage of therapeutic compound released, and the X-axis represents time.

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

What is claimed:

1. A pharmaceutical composition comprising famciclovir and a release retardant wherein said composition comprises at least 5% by weight release retardant.

2. The pharmaceutical composition of claim 1, comprising at least 60% by weight famciclovir.

3. The pharmaceutical composition of claim 1, wherein said composition has a modified release dissolution profile.

4. The pharmaceutical composition of claim 1, wherein said release retardant is a polymer.

5. The pharmaceutical composition of claim 4, wherein famciclovir has a melting point of approximately 104° C., and said polymer has a glass transition temperature less than said melting point.

6. The pharmaceutical composition of claim 5, further comprising a plasticizer.

7. The pharmaceutical composition of claim 1, wherein said release retardant is a non-polymeric release retardant.

8. The pharmaceutical composition of claim 7, wherein famciclovir has a melting point of approximately 104° C., and wherein said non-polymeric release retardant has a melting point less than said melting point.

9. The pharmaceutical composition of claim 1, wherein said composition includes from 500-1500 mg famciclovir.

10. The pharmaceutical composition of claim 4, wherein said polymer is a mixture of polyvinyl acetate and polyvinylpyrrolidone.

11. A method of making a modified release pharmaceutical composition comprising the step of granulating famciclovir with a release retardant in a extruder while heating to a temperature below 104° C. to form granules.

12. The method of claim 11, further comprising compressing the granules into a tablet.

13. The method of claim 11, wherein said extruder is a twin-screw extruder.

14. The method of claim 11, wherein the release retardant is a polymer.

15. The method of claim 14, wherein said polymer is a mixture of polyvinyl acetate and polyvinylpyrrolidone.

16. The method of claim 11, wherein said modified release pharmaceutical composition comprises at least 5% release retardant by weight of the composition.

17. A method of making a modified release pharmaceutical composition comprising the step of granulating famciclovir with a polymer in a extruder while heating to a temperature between the T_{g} of said polymer and below the melting range of famciclovir to form granules.
